(19) United States
(12) Patent Application Publication
(10) Pub. No.: US 2024/0218222 A1
(43) Pub. Date: Jul. 4, 2024

(54) SYNTHETIC HYBRID SPIDROIN-AMYLOID-MUSSEL FOOT PROTEIN FOR UNDERWATER ADHESION OF DIVERSE SURFACES

(71) Applicant: Washington University, St. Louis, MO (US)

(72) Inventors: Fuzhong Zhang, St. Louis, MO (US); Eugene Kim, St. Louis, MO (US)

(73) Assignee: Washington University, St. Louis, MO (US)

(21) Appl. No.: 18/043,330

(22) PCT Filed: Aug. 25, 2021
(86) PCT No.: PCT/US21/47593

§ 371 Date: Feb. 27, 2023

Related U.S. Application Data
(60) Provisional application No. 63/069,987, filed on Aug. 25, 2020.

(51) Int. Cl. C09J 189/00; A61L 24/10; C07K 14/435; C09J 9/00

(52) U.S. Cl. CPC C09J 189/00; A61L 24/108; C07K 14/43504; C07K 14/43518; C09J 9/00; C07K 2319/00

(57) ABSTRACT

Hybrid proteins are described that include an amino acid sequence with at least two zipper-forming protein sequences and at least one flexible sequence from a spider silk protein sequence, in which each of the at least one flexible sequences is positioned between each pair of the at least two zipper-forming protein sequences to form a repeated sequence. The amino acid sequence also includes at least one mussel foot protein sequence positioned at a C-terminus or an N-terminus of the alternating repeated sequence. Also described are adhesive hydrogels and methods of forming the adhesive hydrogels using the hybrid proteins described herein.

### CROSS-REFERENCE TO RELATED APPLICATIONS

This application is a US national application of PCT International Application No. PCT/US21/47593, filed on Aug. 25, 2021. PCT International Application No. PCT/US21/47593 claims priority from U.S. Provisional Application Ser. No. 63/069,987 filed on Aug. 25, 2020, which is incorporated herein by reference in its entirety.

### STATEMENT REGARDING FEDERALLY FUNDED RESEARCH AND DEVELOPMENT

This invention was made with government support under N000141912126 awarded by the Office of Naval Research. The government has certain rights in the invention.

### MATERIAL INCORPORATED-BY-REFERENCE

The Sequence Listing, which is a part of the present disclosure, includes a computer-readable ASCII file entitled “019481-WO-US_SEQ_ST25.txt” (3621 bytes, created on Mar. 4, 2024) comprising nucleotide and/or amino acid sequences of the present invention. The subject matter of the Sequence Listing is incorporated herein by reference in its entirety.

### BACKGROUND OF THE DISCLOSURE

The field of the disclosure relates generally to adhesive compositions. More specifically, the field of the disclosure relates to adhesive compositions that include hydrogels formed using fusion proteins.

Engineering strong adhesives that work underwater remains a challenging feat in material design, but makes possible numerous practical applications ranging from underwater repairs of pipes, vessels, and equipment, to surgical glues for various biological tissues. The challenge largely arises from the need for strong bonding to diverse types of surfaces in aqueous environments, as well as providing a cohesive network of interactions within the glue to hold two surfaces together while absorbing energy during detachment. Many commercially available glues, such as epoxy glues, adhere to surfaces based on nucleophilic reactions with surface functional groups, forming covalent bonds. While strong adhesion can be obtained in dry conditions using epoxy-type glue compositions, such compositions fail to work on wet surfaces due to nucleophilic attack from water molecules and an inability to penetrate surface hydration layers. Recent biomimetic materials inspired by natural underwater adhesive proteins, such as those from mussels, barnacles, and sandcastle worms, have obtained substantial success in underwater surface adhesion. These materials utilize a collection of weak but water-insensitive noncovalent interactions that enhance underwater adhesion. Such interactions include catechol-mediated bidentate hydrogen bonds and cation-π interactions, phosphate-mediated electrostatic interactions, and hydrophobic interactions to different surfaces. However, biomimetic glues typically suffer from weak cohesive strength, leading to cohesive failure, in which the glue remains stuck to both sides of the adhering surfaces with little bonding force between the two bulk surfaces, limiting their practical applications. An ideal underwater glue would thus require careful tuning of the molecular interaction networks to balance both cohesive and adhesive interactions. While too weak of a cohesive interaction leads to cohesive failure, too strong of a cohesive interaction makes the glue brittle and reduces surface adhesion.

### BRIEF DESCRIPTION OF THE DISCLOSURE

In one aspect, a hybrid protein is disclosed that includes an amino acid sequence with at least two zipper-forming protein sequences and at least one flexible sequence from a spider silk protein sequence. Each of the at least one flexible sequences is positioned between each pair of the at least two zipper-forming protein sequences to form a repeated sequence. The amino acid sequence also includes at least one mussel foot protein sequence. Each of the mussel foot proteins may be positioned at a C-terminus or an N-terminus of the alternating repeated sequence.

In another aspect, an adhesive hydrogel is disclosed that includes the hybrid proteins described herein.

In an additional aspect, a method of producing the hybrid protein described herein is disclosed that includes culturing a host cell comprising at least one fusion gene sequence encoding the hybrid protein.

In another additional aspect, a method of forming the adhesive hydrogel described herein is disclosed. The method includes reconstituting a lyophilized hybrid protein in a solvent to form a protein solution, contacting an ionic solution dropwise with the protein solution to form a mixture, and culturing the mixture to form the adhesive hydrogel.

In other additional aspects, an underwater adhesive composition and a surgical glue comprising the adhesive hydrogel described herein are disclosed.

In yet another additional aspect, a method of joining at least two adherend materials is disclosed that includes positioning the adhesive hydrogel described herein between the at least two adherend materials and maintaining the position of the adhesive hydrogel to form a plurality of bonds between the adhesive hydrogel and the at least two adherend materials.

Other objects and features will be in part apparent and in part pointed out hereinafter.

### BRIEF DESCRIPTION OF THE DRAWINGS

The following drawings illustrate various aspects of the disclosure.

FIG. 1A is a schematic diagram illustrating the design (right) and production (left) of a hybrid protein 8×KLV-Mfp5 in accordance with one aspect of the disclosure.

FIG. 1B is a schematic diagram illustrating the cohesive and adhesive interactions associated with bulk adhesion of the hybrid protein of FIG. 1A with various surfaces.

FIG. 2A is a surface morphology SEM image of an 8×KLV hybrid protein hydrogel in accordance with one aspect of the disclosure.

FIG. 2B is a graph summarizing FTIR spectra of the 8×KLV hybrid protein hydrogel of FIG. 2A.

FIG. 2C is a surface morphology SEM image of the 8×KVL-Mfp5 hybrid protein hydrogel in accordance with one aspect of the disclosure.

FIG. 2D is a graph summarizing FTIR spectra of the 8×KVL-Mfp5 hybrid protein hydrogel of FIG. 2C.

FIG. 3 is a graph summarizing stress-strain curves for ultimate tensile strength at fracture for the 8×KLV-Mfp5 hydrogel, mechanical hysteresis after consecutive cycles of loading-unloading of the 8×KLV-Mfp5 hydrogel, and ultimate tensile strength of the 8×KLV hydrogel.

FIG. 4A contains a graph summarizing the underwater adhesion strength of 8×KLV and 8×KLV-Mfp5 hydrogels on aluminum surfaces.

FIG. 4B contains a graph summarizing the underwater adhesion strength of 8×KLV-Mfp5 hydrogel on a glass surface.

FIG. 4C contains a graph summarizing the underwater adhesion strength of 8×KLV-Mfp5 on polycarbonate (PC), polyethylene (PE), polymethylmethacrylate (PMMA), and polyvinylchloride (PVC) surfaces measured using a single-lap shear adhesion test.

FIG. 4D contains a graph summarizing the underwater adhesion strength of 8×KLV-Mfp5 hydrogel on porcine skin and bovine tendon surfaces measured using a single-lap shear adhesion test.

FIG. 5A is an SEM image of the 8×KLV-Mfp5 hydrogel obtained after pH-induced oxidation treatment.

FIG. 5B is a graph summarizing the underwater adhesion strength of 8×KLV-Mfp5 hydrogel under normal conditions, after curing post-pH-induced oxidation treatment, after pH-induced oxidation treatment post-curing, and after iron-induced crosslinking post-curing (n.d.=not detectable).

Those of skill in the art will understand that the drawings, described below, are for illustrative purposes only. The drawings are not intended to limit the scope of the present teachings in any way.

### DETAILED DESCRIPTION OF THE DISCLOSURE

Artificially designed, hybrid proteins made using synthetic biology approaches present a new and attractive class of materials for a variety of different purposes. Nature has evolved many protein-based, high-performance materials, e.g. spider silk, dragonfly resilin, and human titin and collagen. These materials confer extraordinary properties and functions, such as high tensile strength and high elasticity, beyond the reach of synthetic organic materials. By incorporating different protein motifs, hybrid proteins can combine multiple favorable structural and functional features of natural and artificial proteins into one protein molecule, resulting in high-performance, multi-functional materials for various applications. DNA-templated biosynthesis of hybrid proteins allows for the control of functional groups at an amino acid resolution for fine-tuning a material's chemical, biological, and mechanical properties.

Furthermore, modern synthetic biology approaches have provided for the incorporation of non-canonical amino acids into protein sequences, largely expanding their functions. Microbial production of natural high-performance materials offers an attractive route for their synthesis, which is sustainable, environmentally friendly, and independent of natural supplies. Recent advances in synthetic biology have made possible the production of not only natural high-performance proteins but also artificially designed proteins and proteins with unnatural amino acids or post-translational modifications, largely expanding the portfolio of protein-based materials.

Adhesive materials have gained much popularity in a variety of applications, such as in biomedicine and repair. However, many commercial adhesives and those recently discovered exhibit poor mechanical properties, confer poor adhesion especially in wet conditions, and are generally limited in application, in part due to a lack of tunability of these materials in various situations. Protein-based adhesives have recently been of particular interest because they can be recombinantly engineered with high tunability, can be produced in high yields, and have shown the potential to be stronger than existing synthetic materials.

In various aspects, a fusion protein is disclosed that includes zipper-forming domains found in human amyloid protein, spidroin protein, and mussel foot protein. The disclosed zipper protein self-assembles into a robust adhesive hydrogel that is mechanically strong, is highly elastic, and is adhesive in wet conditions. The disclosed hydrogel adhesive material has an ultimate tensile strength of ˜1 MPa and can cure underwater and adhere materials to up to ˜1 MPa in adhesion strength. Due to the nature of the domains used in this fusion protein, the hydrogel can be treated to allow for debonding under oxidizing and iron-chelating conditions. This study provides an easy strategy in synthetic biology to design and produce tough adhesive materials from tunable components that are found in nature.

The disclosed hybrid proteins and associated hydrogels represent a new class of artificially designed, hybrid proteins and multi-functional underwater adhesives for diverse surfaces. The disclosed hybrid proteins and associated hydrogels combine the attractive properties of multiple natural protein domains—the ability to self-assemble into stable β-sheets of an Aβ zipper-forming domain, the flexible nature of the spider silk's amorphous domain, and the underwater adhesivity of Mfp5—into one hybrid protein, resulting in a strong, tough, and adhesive material. These properties mostly originate from the repetitive, primary peptide sequences, which allow for multi-scale assembly into higher-ordered structures. This hybrid protein design opens the possibility to tune each property of the resulting material through corresponding protein domains. With a better understanding of the protein sequence-structure-property relationship, mutations can easily be made using the biosynthetic methods described herein to improve material properties. Furthermore, from the vast variety of natural protein domains, the materials and methods described herein may facilitate the design of a wide range of hybrid proteins with properties consistent with high-performance, multifunctional materials.

Compared to existing underwater adhesives made of organic polymers, the protein-based hydrogel adhesive is not only fabricated from a more sustainable process but also offers comparable or stronger underwater adhesion. Additionally, the hydrogel has a negative buoyancy in water and allows for facile application in underwater conditions, which is particularly advantageous compared to liquid adhesives. The disclosed underwater adhesive design takes advantage of a semi-crystalline material approach. The crystalline domain from Aβ effectively prevents Mfp5 aggregation, promoting intermolecular interactions between amorphous Mfp5 sequences and leading to a strong and tough hydrogel, where 8×KLV or Mfp5 alone cannot form. The adhesive hydrogel adheres strongly to a variety of surfaces, including chemically inert plastics and heterogeneous mammalian tissue surfaces. The strong adhesion, controllable debonding, and biodegradability make the hydrogel an attractive candidate for applications in surgery and underwater repair.

### Hybrid Protein

“Fusion gene”, as used herein, refers to a gene formed through the joining of two or more genes that originally coded for separate proteins.

“Recombinant fusion genes”, as used herein, refers to fusion genes created artificially by recombinant DNA technology for use in biological research or therapeutics. Recombinant fusion genes encode hybrid proteins made of polypeptides having different functions or physico-chemical patterns.

“Fusion protein” or “hybrid protein”, as used herein, refers to a protein created by the translation of a fusion gene, resulting in single or multiple polypeptides with functional properties derived from each of the original separate proteins.

The disclosed fusion protein is configured to self-assemble into a semi-crystalline protein hydrogel that contains both structured and amorphous domains protein, resulting in a material with strong cohesion characterized by both high strength and toughness. In various aspects, the fusion protein includes a plurality of zipper-forming sequences from an amyloid protein, a plurality of flexible sequences from a dragline spider silk protein, and at least one sequence from a mussel foot protein. In these various aspects, each zipper forming sequence is linked by at least one dragline spider silk protein sequence in a repeated pattern, and the mussel foot protein sequence is attached at the protein's C-terminus and/or N-terminus.

Without being limited to any particular theory, the zipper-forming sequences are characterized by a strong tendency to self-assemble into stable β-crystals under aqueous conditions. In various aspects, any suitable zipper forming sequence may be selected for use in the disclosed fusion protein without limitation. Non-limiting examples of suitable zipper-forming sequences include at least a human prion₁₇₀₋₁₇₅ sequence (SNQNNF, SEQ ID NO:1), an αβ-crystallin₉₅₋₁₀₀ sequence (GDVIEV, SEQ ID NO:2), an islet amyloid polypeptide₂₃₋₂₉ sequence (FGAILSS, SEQ ID NO:3) an Aβ-amyloid₁₆₋₂₂ sequence (KLVFFAE, SEQ ID NO: 4), and any fragment thereof. In one aspect, the disclosed fusion protein comprises the zipper-forming sequence from Aβ amyloid protein comprising an Aβ-amyloid₁₆₋₂₂ sequence (KLVFFAE, SEQ ID NO: 4) or any fragment or variant thereof.

In various aspects, the hybrid protein may include any number of repeated zipper-forming sequences without limitation. In some aspects, the number of repeated zipper-forming sequences is at least one zipper-forming sequence, at least two zipper-forming sequences, at least three zipper-forming sequences, at least four zipper-forming sequences, at least five zipper-forming sequences, at least six zipper-forming sequences, at least seven zipper-forming sequences, at least eight zipper-forming sequences, at least nine zipper-forming sequences, at least ten zipper-forming sequences, or more. In one aspect, the hybrid protein includes eight repeats of the zipper-forming sequence from an Aβ amyloid protein comprising β-amyloid₁₆₋₂₂ (KLVFFAE, SEQ ID NO: 4) or any fragment or variant thereof.

In various aspects, flexible sequences from dragline spider silk protein connect multiple zipper-forming sequences in the disclosed hybrid protein. Without being limited to any particular theory, the flexible sequences are thought to bridge between the zipper-forming sequences without disturbing the formation of β-sheets and fibril spines. Spider silk is characterized by high strength and toughness derived from a high molecular weight spidroin protein with more than 100 tandem repeats of polyalanine and glycine-rich sequences in which the polyalanine sequences form extensive β-sheet nanocrystals linked together by amorphous, glycine-rich domains. In the hybrid protein, the intrinsically disordered sequence formed by alternating zipper-forming sequences and flexible dragline spider silk protein sequences facilitates the formation of β-crystals and amorphous domains.

Any suitable flexible sequence from a spider silk protein may be used in the hybrid protein without limitation. Non-limiting examples of suitable flexible sequences from spider silk proteins include a glycine-rich sequence from Nephila clavipes MaSp1 dragline spidroin (GGAGQGG-YGGLGSQGTSGRGGLGGQGAG, SEQ. ID. 5) and any fragment or variant thereof.

To endow the hybrid protein with enhanced surface adhesion properties, a sequence from a mussel foot protein may be included at the C-terminus or N-terminus of the amino acid sequence. Without being limited to any particular theory, mussel foot proteins (Mfp) have strong underwater adhesiveness and flexibility derived from varying levels of 3,4-dihydroxyphenylalanine (DOPA), which arise from post-translational modification of tyrosine residues by tyrosinases, a group of natively expressed hydroxylating enzymes. DOPA is known to form adhesive protein-surface interactions via bidentate hydrogen bonding, metal complexation, and hydrophobic interactions as well as cohesive protein—protein interactions via bis- and tris-DOPA-Fe3+ complexation, DOPA—DOPA hydrogen bonding, aryloxyl radicalization, π-π stacking interactions, and cation-π interactions.

Any suitable adhesive sequence from any mussel foot protein may be included in the disclosed hybrid protein without limitation. Non-limiting examples of suitable adhesive sequences from mussel foot proteins include mussel foot protein-3 Mfp3 (ADYYGPKYGPPRRYGGGNYNRYGRRYGGYKGWNNGWKRGRWGRKYY, SEQ ID NO:6)), mussel foot protein-5 Mfp5 (SSEEYKGGYYPGNAYHYSGGSYHGSGYHGGYKGKYYGKAKKYYYKYKNSGKYKY LKKARKYHRKGYKYYGGSS, SEQ ID NO:7), mussel foot protein-6 Mfp6 (GGGNYRGYCSNKGCRSGYIFYDNRGFCKYGSSSYKYDCGNYACLPRNPYGRVKYY CTKKYSCPDDFYYYNNKGYYYYNDKDYGCFNCGSYNGCCLRSGY, SEQ ID NO:8), and any fragment or variant thereof. In some aspects, the disclosed hybrid protein includes at least one Mytilus galloprovincialis mussel foot protein-5 (Mfp5) attached at the C-terminus and/or N-terminus. In one aspect, the disclosed hybrid protein includes a Mytilus galloprovincialis mussel foot protein-5 (Mfp5) attached at the C-terminus.

In various aspects, the hybrid protein includes DOPA substituted for at least a portion of the tyrosine residues within the protein's amino acid sequence. In various aspects, at least 10%, at least 20%, at least 30%, at least 40%, at least 50%, at least 60%, at least 70%, at least 80%, at least 90%, and at least 90% of the tyrosine residues within the protein's amino acid sequence are substituted with DOPA moieties. Without being limited to any particular theory, the DOPA moieties enhance the adhesion strength, materials strength, and/or material toughness by adhesive protein-surface interactions and protein-protein interactions as described above.

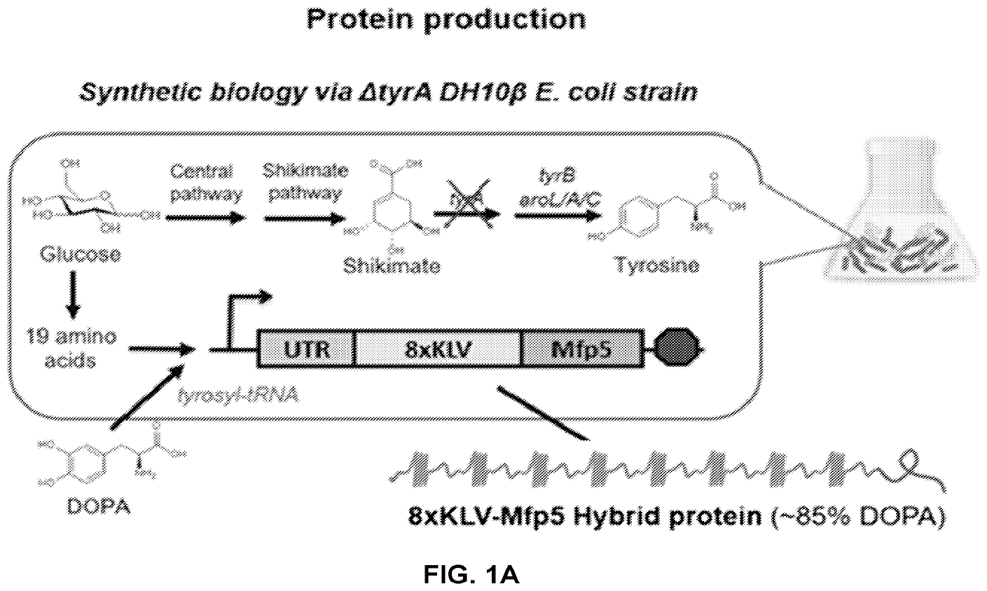

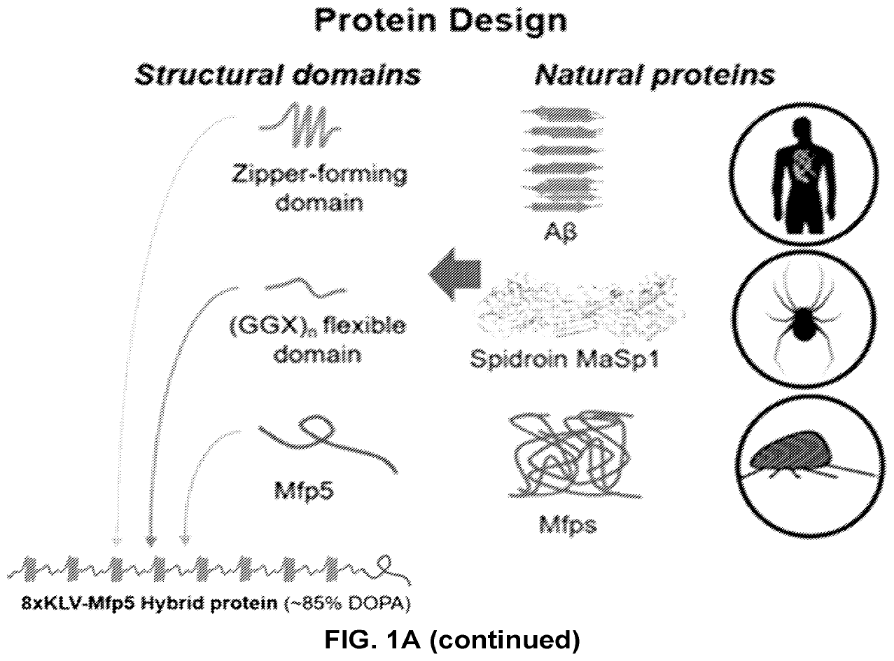

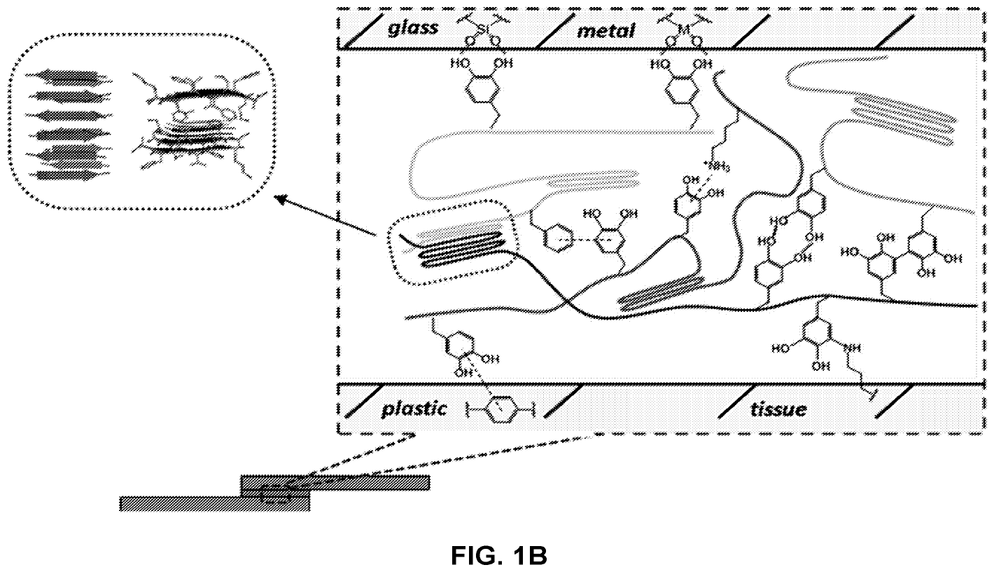

An illustration of the hybrid protein in one aspect is illustrated schematically in FIG. 1A in one aspect. As illustrated in FIG. 1A, the hybrid protein 8×KLV-Mfp-5 includes an alternating series of eight Aβ amyloid protein (KLV) sequences and eight spider drag silk sequences, as well as a Mytilus galloprovincialis mussel foot protein-5 (Mfp5) attached at the C-terminus of the protein. Without being limited to any particular theory, the repeated sequences were repeated 8 times to obtain a sufficient chain length for promoting intermolecular interactions, which contributes to both material strength and toughness. The inclusion of the disordered Mfp5 chain provides for intermolecular interactions between hybrid proteins using DOPA and other functional groups, forming additional amorphous domains and providing toughness to the material.

### Biosynthesis of Fusion Protein

In various aspects, the disclosed hybrid protein is produced from synthetic DNA by an engineered Escherichia coli strain that stably expresses repetitive sequences. The hybrid proteins may be produced by biosynthesis using any suitable recombinant host cell that includes the fusion genes encoding the hybrid protein without limitation. In some aspects, the host cells are E. coli cells including E. coli strain NEB10β and variants thereof.

In various aspects, the biosynthesized hybrid protein includes 3,4-dihydroxyphenylalanine (DOPA) moieties substituted at tyrosine residues as described above to enhance the adhesive strength, material strength, and material toughness of the hybrid proteins ad associated hydrogels. Any suitable method may be used to substitute the DOPA moieties into the tyrosine residues of the hybrid protein without limitation. In some aspects, the DOPA moieties may be substituted by post-translational modification of the tyrosine residues using tyrosinases. In other aspects, the incorporation of DOPA may be included within the biosynthesis method as described below.

In various other aspects, the tyrA gene may be deleted from the host cell. The protein product of the tyrA gene catalyzes the first step in tyrosine biosynthesis. Fermenting tyrA⁻ host cells in a tyrosine-depleted growth medium supplementation with DOPA causes mis-aminoacylation of DOPA to endogenous tyrosyl tRNAs, causing the incorporation of DOPA into the hybrid protein at tyrosine codons. In one aspect, the host cell may comprise E. coli strain NEB 10β ΔtyrA engineered from the NEB 10β strain by deleting the tyrA gene using homologous recombination. In various aspects, this biosynthetic strategy may produce hybrid proteins with DOPA incorporation efficiencies as high as about 90%.

In various aspects, the disclosed method of producing the hybrid proteins provides the ability to vary specific amino acid sequences, relative compositions of amino acid motifs, or relative orders of these motifs to module at least one or more properties of the hybrid proteins and/or associated adhesive hydrogels including, but not limited to high tensile strength, elasticity, resilience, and adhesion strength. Better understanding the material sequence-structure-function relationship using the synthetic biology methods described above may lead to the synthesis of superior designer glues comparable or superior to existing adhesive compositions.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site, all other sequences of the gene and its controlling regions can be numbered. Downstream sequences (i.e., further protein-encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects the expression of the coding DNA sequence (i.e., that the coding sequence or functional RNA is under the transcriptional control of the promoter). Coding sequences can be operably linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. The amino acid sequence can be modulated with the help of computer simulation programs well-known in the art that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log ₁₀[Na⁺])+0.41(fraction G/C content)−0.63(% formamide)−(600/1). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| Side Chain Characteristic | Amino Acid |
|---|---|
| Non-polar (hydrophobic) | |
| A. Aliphatic: | A L I V P |
| B. Aromatic: | F W |
| C. Sulfur-containing: | M |
| D. Borderline: | G |
| Uncharged-polar | |
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Substitutions III

| Original | Exemplary |
|---|---|
| Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Adhesive Hydrogel

In various aspects, the disclosed hybrid protein may form an adhesive hydrogel with adhesive strength, material strength, and material toughness as described below. Without being limited to any particular theory, the repeated amyloid zipper-forming sequences within the hybrid protein self-assemble under conditions described below, triggering an extensive intermolecular interaction network and transforming the hybrid protein into a strong and tough hydrogel that exhibits enhanced underwater adhesion via surface-exposed DOPA residues. The DOPA residues enhance the adhesion of the hydrogel to a variety of surfaces including, but not limited to glass, metal, plastic, and biological tissues.

In various aspects, the adhesive hydrogel may be formed by incubating the hybrid protein within a phosphate-buffered saline solution that includes a concentration of sodium phosphate. In some aspects, the hybrid proteins may be reconstituted by dissolving purified, lyophilized proteins in a solvent. Any suitable solvent may be used to form the hybrid protein solution including, but not limited to, dimethyl sulfoxide (DMSO), dimethylformamide (DMF), acetic acid, acetonitrile, methanol, propanol, isopropanol, trifluoroethanol (TFE), trichloromethane (TCM), dichloromethane (DCM), and 1,1,1,3,3,3-hexafluoroisopropanol (HFIP). In one aspect, the hybrid protein solution is formed by dissolving the purified, lyophilized proteins in HFIP.

In various aspects, the protein solution may be provided at any suitable concentration without limitation. In various aspects, the protein solution may be provided at a protein concentration of at least 0.5% w/v, at least 1% w/v, at least 2% w/v, at least 3% w/v, at least 4% w/v, at least 5% w/v, at least 6% w/v, at least 7% w/v, at least 8% w/v, at least 10% w/v, at least 15% w/v, and at least 20% w/v or more. In one aspect, the protein solution may be provided at a protein concentration of about 7.5% w/v. In some aspects, the protein may be dissolved into an excess of solvent at a relatively low protein concentration, followed by the removal of excess solvent to obtain the desired protein concentration within the protein solution. Any suitable method of solvent removal may be used to increase the initial concentration of protein in the protein solution including, but not limited to, evaporation.

In various aspects, the protein solution may be contacted with an ionic solution to form the adhesive hydrogel. In some aspects, the ionic solution is added dropwise to the protein solution, and the resulting mixture is incubated for an extended period including, but not limited to, at least 12 hours. Any ionic solution may be used to form the hydrogel including, but not limited to, phosphate-buffered saline solution that includes sodium phosphate and sodium chloride. In various aspects, the phosphate-buffered saline solution may include a sodium phosphate concentration ranging from about 5 mM to about 150 mM. In various other aspects, the phosphate-buffered saline solution may include a sodium phosphate concentration ranging from about 1 mM-10 mM, from about 5 mM to about 15 mM, from about 10 mM to about 20 mM, from about 15 mM to about 30 mM, from about 20 mM to about 40 mM, from about 30 mM to about 50 mM, from about 40 mM to about 60 mM, from about 50 mM to about 70 mM, from about 60 mM to about 80 mM, from about 75 mM to about 125 mM, from about 100 mM to about 150 mM, from about 125 mM to about 175 mM, and from about 150 mM to about 200 mM or more. In one aspect, the ionic solution added dropwise to the protein solution is phosphate-buffered saline containing 100 Mm sodium phosphate and 300 mM sodium chloride.

Without being limited to any particular theory, any one or more of a plurality of factors may impact the properties of the adhesive hydrogel formed as described above including, but not limited to, ionic strength of the ionic solution, protein concentration within the protein solution, and solvent composition in the protein solution. Non-optimal combinations of factors may lead to protein precipitation or gels that are too soft. In various aspects, a combination of factors may be selected to form firm and opaque hydrogels, indicating the formation of a stable network within the hydrogel.

In various aspects, the hydrogel solution may be formed into any desired shape by placing the newly-formed protein/ionic solution mixture into a mold. In some aspects, the molded hydrogel may be further shaped by cutting, trimming, or any other suitable shaping method without limitation. The mold may be formed using any suitable material without limitation including, but not limited to, PDMS. In some aspects, the cured hydrogel may be further incubated in an excess of a solution including, but not limited to, PBS to leach out excess solvent from the protein solution used to form the hydrogel, such as HFIP.

In various aspects, the adhesive hydrogels exhibit enhanced material properties relative to existing adhesive compositions, in particular with respect to underwater adhesion strengths. Without being limited to any particular theory, the enhanced material properties are thought to result from the semi-crystalline material structure of the adhesive hydrogels that contain both structured and unstructured domains as described above and as illustrated in FIG. 1B. In various aspects, the cured hydrogel has a density that is greater than water, thus facilitating the use of the hydrogel as an underwater adhesive composition.

Illustrative examples of the material properties of the 8×KLV-Mfp5 hydrogel are described in additional detail in the examples below. In some aspects, the adhesive hydrogels have an ultimate tensile strength of approximately 1.0 MPa or more and an adhesion strength as high as about 1.0 MPa or more, depending on the adhesion surface.

In addition, the adhesive hydrogels have relatively high elasticity that provides the ability to stretch reversibly to strains up to about 300% or higher. In various aspects, the underwater adhesion strength is at least 10 kPa, at least 20 kPa, at least 25 kPa, at least 30 kPa, at least 35 kPa, at least 40 kPa, at least 45 kPa, at least 50 kPa, at least 75 kPa, at least 100 kPa, at least 150 kPa, at least 200 kPa, at least 250 kPa, at least 300 kPa, at least 100 kPa, at least 350 kPa, at least 400 kPa, at least 450 kPa, at least 500 kPa, at least 550 kPa, at least 600 kPa, at least 650 kPa, at least 700 kPa, at least 750 kPa, at least 800 kPa, at least 850 kPa, at least 900 kPa, or at least 1 MPa or more, depending on the adherend surface. On an aluminum surface, the underwater adhesion strength is about 250 kPa or more in one aspect. On a glass surface, the underwater adhesion strength is about 1 MPa or more in one aspect. On a plastic surface, the underwater adhesion strength ranges from about 100 kPa to about 250 kPa or more in one aspect. On skin, the underwater adhesion strength is about 75 kPa or more in one aspect. On tendon, the underwater adhesion strength is about 35 kPa or more in one aspect.

In other aspects, the adhesive hydrogels have an initial damping capacity of about 220 KJ/m³ or more, and a steady-state damping capacity in subsequent loading cycles of about 70 KJ/m³ or more. In various other aspects, the adhesive hydrogels have an initial damping capacity of at least 20 KJ/m³, at least 40 KJ/m³, at least 60 KJ/m³, at least 80 KJ/m³, at least 100 KJ/m³, at least 120 KJ/m³, at least 140 KJ/m³, at least 160 KJ/m³, at least 180 KJ/m³, at least 200 KJ/m³, at least 220 KJ/m³, at least 240 KJ/m³, at least 260 KJ/m³, at least 280 KJ/m³, and at least 300 KJ/m³ or more. In other additional aspects, the adhesive hydrogels have a steady-state damping capacity in subsequent loading cycles of at least 10 KJ/m³, at least 20 KJ/m³, at least 30 KJ/m³, at least 70 KJ/m³, at least 70 KJ/m³, at least 70 KJ/m³, at least 70 KJ/m³, at least 70 KJ/m³, at least 40 KJ/m³, at least 50 KJ/m³, at least 60 KJ/m³, at least 70 KJ/m³, at least 80 KJ/m³, at least 90 KJ/m³, at least 100 KJ/m³, at least 110 KJ/m³, at least 120 KJ/m³, at least 130 KJ/m³, at least 140 KJ/m³, at least 150 KJ/m³, and at least 200 KJ/m³ or more. Without being limited to any particular theory, the enhanced damping capacity dissipates large amounts of energy created during detaching, resulting in enhanced material toughness.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Any publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

### Methods of Using Adhesive Hydrogels

In various aspects, the hybrid protein-based adhesive hydrogels described herein may be used as underwater adhesives to join a variety of surfaces including, but not limited to, metal surfaces, glass surfaces, plastic surfaces, and biological tissues such as skin and tendon.

In various aspects, the cured adhesive hydrogel formed as described above may be positioned between two or more adherend surfaces and held in place to allow time for the DOPA moieties of the hydrogel to bond with various functional groups exposed on the adherend surfaces, as illustrated in FIG. 1B. In some aspects, the cured adhesive hydrogels may be used in biomedical applications including, but not limited to, surgical glues, due to their potential biocompatibility and biodegradability. In some aspects, the adhesive hydrogels may be used as surgical glues in tendon-bone repairs. The use of the adhesive hydrogels in tendon-bone repairs overcome at least some of the limitations of existing tendon-bone repairs such as sutures that typically have high failure rates and necessitate puncturing healthy tissue.

In various additional aspects, the adhesive hydrogels may be detached from an adherend surface by exposing the hydrogel and/or the adherend surface to an oxidizing or chelating solution to disrupt the bonding of catechols of the DOPA moieties to the adherend surface, such as catechol-metal coordination and catechol-silica bidentate hydrogen bonding interactions. Any suitable oxidizing solution may be used to detach the adhesive hydrogel from the adherend surface without limitation. Non-limiting examples of suitable oxidizing solutions include FeCl₃ solution, pH 11 buffer, and any other suitable oxidizing solution. In one aspect, the oxidizing solution may be a 1 mM FeCl₃ solution.

### EXAMPLES

The following Examples describe or illustrate various embodiments of the present disclosure. Other embodiments within the scope of the appended claims will be apparent to a skilled artisan considering the specification or practice of the disclosure as described herein. It is intended that the specification, together with the Examples, be considered exemplary only, with the scope and spirit of the disclosure being indicated by the claims, which follow the Examples.

### EXAMPLE 1: Production of Hybrid Proteins 8×KLV and 8×KLV-Mfp5

To demonstrate the production of hybrid proteins 8×KLV and 8×KLV-Mfp5 using recombinant host cells, the following experiments were conducted.

Unless otherwise noted, all chemicals and reagents were obtained from Millipore Sigma (Saint Louis, MO, USA). Plasmid purification and gel extraction kits were purchased from iNtRON Biotechnology (Seoul, South Korea). FastDigest restriction enzymes and T4 DNA ligase were purchased from Thermo Fisher Scientific (Austin, TX, USA) and were used according to suggested protocols from the manufacturer.

Plasmid construction was accomplished using an SI-Brick system was to facilitate the cloning of hybrid proteins. Specifically, DNA coding for the 8×KLV protein was cloned by digesting plasmid KLV-1X with restriction enzymes NheI and BcuI and performing sequential ligations, resulting in plasmid pE8a-8×KLV. The Mfp5 gene sequence was obtained by performing a PCR on plasmid pE7a-mfp5(1) with primers BcuI-Mfp5-F and Kpn2I-Mfp5-R e S2).23 The PCR amplicon was digested with restriction enzymes BcuI and Kpn2I and ligated into the pE8a-8×KLV cut with the same restriction sites, yielding plasmid pE8a-8×KLV-Mfp5.

E. coli strain NEB10β ΔtyrA was engineered from the NEB10β strain (Thermo Fisher Scientific, Waltham, MA) by deleting the tyrA gene using homologous recombination. Specifically, the FRT (FLP recognition target)-flanked kanamycin marker was amplified from plasmid pKD13 using primers containing the 5′ and 3′ sequences homologous to the tyrA gene, allowing the deletion of both the tyrA promoter and its entire coding sequence. The PCR amplicon was purified and co-transformed with plasmid pKD46 into the NEB10β competent cell. The transformants were selected on a kanamycin-resistant plate. Deletion of tyrA was confirmed by colony PCR and Sanger sequencing.

E. coli strain NEB10β ΔtyrA was used to express the DOPA-containing 8×KLV-Mfp5, while E. coli strain NEB10β was used for the expression of all other proteins in this experiment. To produce 8×KLV protein, transformed E. coli strain was cultured in shake flasks with Luria-Bertani (LB) broth containing 10 g/L tryptone, 10 g/L sodium chloride, and 5 g/L yeast extract with the appropriate antibiotic (100 μg/mL ampicillin). Fresh transformants were cultivated for 8-16 hours in 5 mL LB medium at 37° C. This culture was then inoculated into 500 mL of LB medium in Erlenmeyer flasks at an initial OD600=0.1. Cultures were grown at 37° C. with shaking to OD600=0.6, then induced by the addition of 0.4% arabinose. The culture was further cultivated at 37° C. at 250 rpm for another 5-7 hours.

To produce DOPA-containing 8×KLV-Mfp5 protein, transformed NEB10β ΔtyrA cells were cultured in shake flasks with Luria-Bertani (LB) broth containing 10 g/L tryptone, 10 g/L sodium chloride, and 5 g/L yeast extract with the appropriate antibiotic (100 μg/mL ampicillin). Fresh transformants were cultivated for 8-16 hours in 5 mL LB medium at 37° C. This culture was then inoculated into 50 mL of M9 media supplemented with 20 canonical amino acids, each at a concentration of 20 mg/L, and cultivated for 8-16 hours. This new culture was then used to inoculate 500 mL of M9 medium with 4 mg/L of tyrosine, and 20 mg/L of the remaining 19 canonical amino acids in Erlenmeyer flasks at an initial OD_{600=0.1}. Cultures were grown at 37° C. with shaking to OD₆₀₀=0.7-0.8. L-DOPA to a final concentration of 1 mM was added to the medium, and cells were induced by the addition of 0.4% arabinose. The culture was further cultivated at 37° C. at 250 rpm for another 5-7 hours.

All cells were harvested by centrifugation at 4,500×g for 20 min at 4° C. Centrifuged cell pellets were either directly extracted or stored at −80° C. until needed.

To purify the protein from the harvested cells, cell pellets were resuspended in 10 mL of guanidine lysis buffer containing 6 M guanidine hydrochloride (BioBasic Inc., Amherst, NY, USA), 50 mM potassium phosphates, and 300 mM sodium chloride at pH 7.4. Cells were lysed by agitation at 250 rpm for 6 hours. Cell lysates were centrifuged at 20,000×g for 20 min at 18° C. To reduce the viscosity, collected lysates were further sonicated on ice (to maintain a roughly ambient temperature) for 30 minutes with a QSonica probe sonicator using 5 seconds on/5 seconds off cycles. The lysates were filtered through 0.2 μm filter membranes. Proteins were purified using an AktaPure Fast Protein Liquid Chromatograph (FPLC, GE Healthcare Inc., Chicago, IL, USA) equipped with a 5 mL nickel affinity chromatography column (GE Healthcare). The column was pre-equilibrated with guanidine lysis buffer followed by sample loading. After washing with 5-10 column volumes (CVs) of guanidine wash buffer (6 M guanidine hydrochloride, 50 mM potassium phosphates, 300 mM sodium chloride, and 50 mM imidazole at pH 7.4), proteins were eluted and fractionated with 5-10 CVs of guanidine elution buffer (6 M guanidine hydrochloride, 50 mM potassium phosphates, 300 mM sodium chloride, and 250 mM imidazole at pH 7.4). For 8×KLV-Mfp5, another purification step using cation exchange chromatography was performed. The eluant from affinity purification was dialyzed in cation exchange buffer (8 M urea, 75 mM sodium acetates, pH 5.5) prior to ion-exchange chromatography. The column was pre-equilibrated with the cation exchange buffer. After protein loading, the column was washed with 5 CVs of wash buffer A (8 M urea, 75 mM sodium acetates, 200 mM guanidine hydrochloride, pH 5.5) and 5 CVs of wash buffer B (8 M urea, 75 mM sodium acetates, 400 mM guanidine hydrochloride, pH 5.5). Proteins were eventually eluted and fractionated with 5-10 CVs of cation exchange elution buffer (8 M urea, 75 mM sodium acetates, 800 mM guanidine hydrochloride, pH 5.5). All eluants were dialyzed in 5% acetic acid followed by 0.5% acetic acid at 4° C. and were then lyophilized.

SDS-PAGE and densitometric analyses were accomplished by casting sodium dodecyl sulfate-polyacrylamide gels in a 1 mm thick casting case (Bio-Rad, Hercules, CA). The gels consisted of a 14% polyacrylamide separating gel, which comprised 14% bisacrylamide, 375 mM tris pH=8.8, 0.1% m/v sodium dodecyl sulfate (SDS), 0.1% m/v ammonium persulfate (APS), and 0.04% v/v tetramethylethlyenediamine (TEMED), as well as a 5% polyacrylamide stacking gel, which comprised 5% bisacrylamide, 125 mM tris pH=6.8, 0.1% m/v SDS, 0.1% m/v APS, and 0.1% v/v TEMED. The gels were run on Mini-PROTEAN Tetra Cells (Bio-Rad) in 1×TGS buffer (30 g/L tris, 144 g/L glycine, and 10 g/L SDS) at 130-150 V for 80-120 minutes, or until the blue dye front exited the gels. Gels were then stained in Coomassie Blue staining solution (40% v/v methanol, 7% v/v acetic acid, 0.1% w/v Coomassie Brilliant Blue) for 30-60 minutes at room temperature with gentle agitation. Gels were then destained in Coomassie Blue destaining solution (40% v/v methanol, 7% v/v acetic acid) for at least 60 minutes. Destain buffer was changed more frequently to remove the Coomassie stain quicker. Gels were imaged on an Azure c600 Imager (Azure Biosystems, Dublin, CA).

Approximately 4 mg of purified, lyophilized protein was transferred into a hydrolysis tube. Liquid phase hydrolysis was performed by dissolving each sample in 200 μL of 6 N HCl and 1% v/v phenol and incubating at 110ºC for 24 hours. A final concentration of 40 μM of norleucine was added to serve as an internal control. The hydrolysis reaction was vortexed and spun down. Lastly, 50 μL was injected into a Hitachi High-Speed Amino Acid Analyzer L-8800 (Hitachi High-Technologies, Schaumburg, IL) and each amino acid was quantified by their corresponding peak area.

### EXAMPLE 2: Preparation and Characterization of 8×KLV and 8×KLV-Mfp5 Hydrogels

To demonstrate the preparation of hydrogels from the 8×KLV and from the DOPA-containing 8×KLV-Mfp5 produced as described in Example 1, the following experiments were conducted.

Purified, lyophilized proteins were fully dissolved in 1,1,1,3,3,3-hexafluoroisopropanol (HFIP) to a protein concentration of 0.5-2.0% w/v in glass vials. Excess HFIP was evaporated to keep a final protein concentration of 7.5% w/v. Phosphate-buffered saline (PBS, 100 Mm sodium phosphates, 300 mM sodium chloride) was added dropwise to the protein/HFIP solution. The solution was covered with parafilm and incubated overnight for at least 12 hours. The parafilm was then removed and the solution was set in ambient conditions quiescently for another 8 hours. Using forceps, the resulting hydrogel was peeled out of the mold and submerged in fresh PBS for at least 12 hours to solvent-exchange any residual HFIP remaining within the hydrogel. The final protein content in the 8×KLV-Mfp5 and 8×KLV hydrogels were 29.4±1.8 w/v % and 35.0±0.69 w/v %, respectively, with water occupying the rest of the materials.

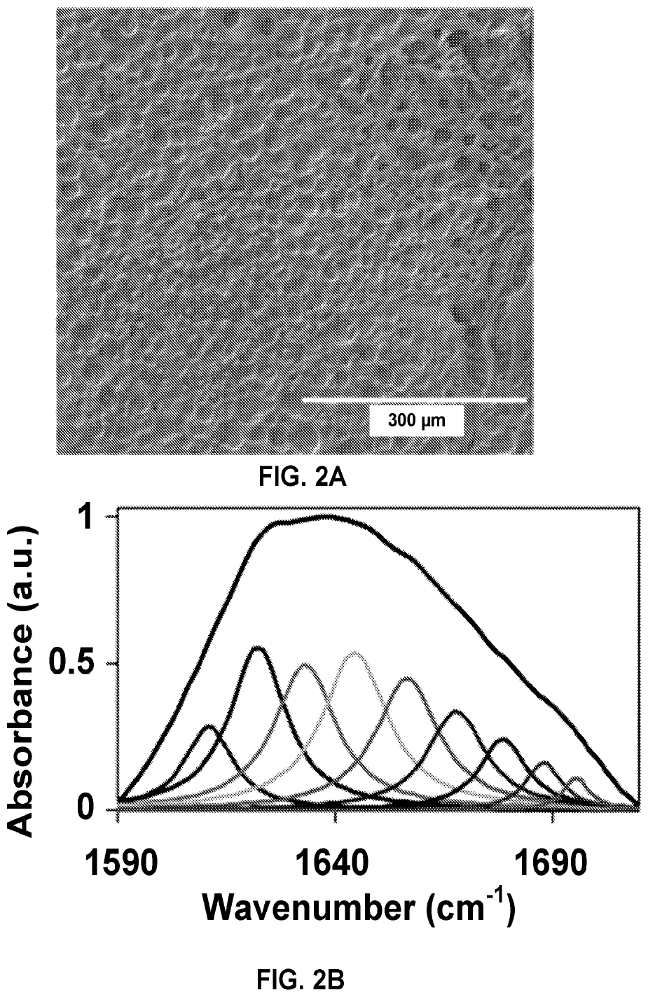

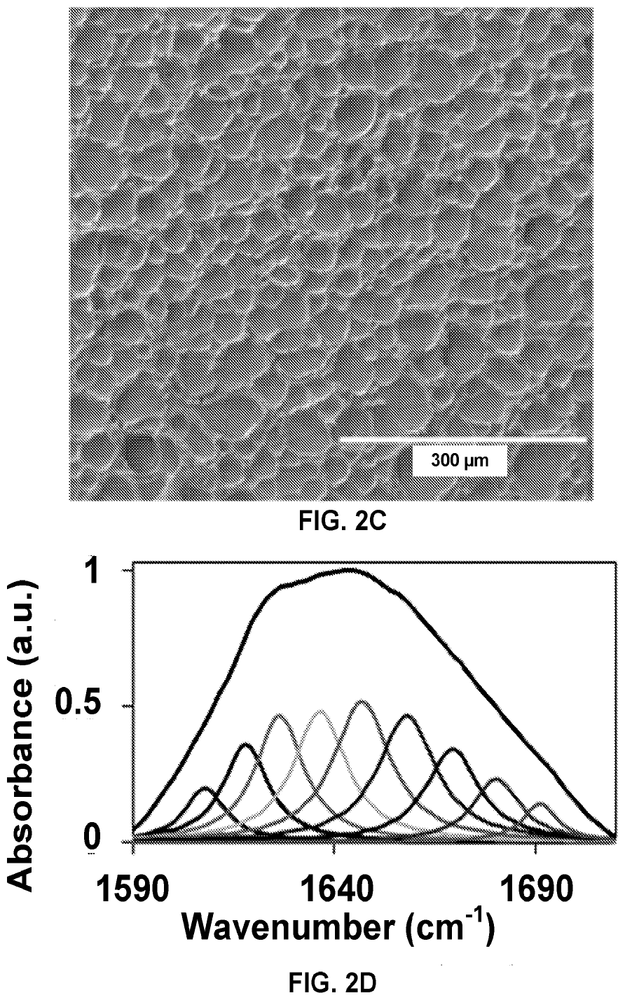

The protein hydrogels were subjected to imaging by scanning electron microscopy (SEM). Protein hydrogels were dried and mounted on a stainless steel sample holder using black carbon tape. The sample was sputter-coated with 10 nm Au using a Leica EM ACE600 high-vacuum sputter coater (Leica Microsystems, Wetzlar, Germany). The coated hydrogels (top-down morphologies and cross-sections) were imaged using a Nova NanoSEM 230 field emission scanning electron microscope (Field Electron and Ion Company, FEI, Hillsboro, Oregon). As shown in FIG. 2A and FIG. 2C, respectively, both 8×KLV-Mfp5 and 8×KLV had rough surfaces with porous structures. Although during hydrogel preparation, the two faces of the hydrogel were in contact with a PDMS mold and air respectively, significant morphological differences between the two faces were not observed.

FTIR was employed to quantitatively determine the number of different types of secondary structures that exist in the protein films produced as described above. Protein secondary structures absorbed IR light in the wavenumber range of 1200 cm⁻¹ and 3600 cm⁻¹. However, because water strongly absorbed IR light in this range, water was removed from the hydrogels prior to FTIR measurement. In order to preserve the morphology of the secondary structures, the hydrogel films were flash-frozen in liquid nitrogen and lyophilized in a freeze dryer (Labconco, Kansas City, MO) overnight. FTIR spectra of the dried hydrogel samples were collected with a Thermo Nicolet 470 FTIR spectrometer (Thermo Fisher Scientific, Waltham, MA) fitted with a Smart Performer ATR accessory with Ge crystal. Spectra were acquired between 400 cm⁻¹ and 4000 cm⁻¹. A total of 128 scans were accumulated and averaged per sample. Averaged spectra were analyzed using Fityk 0.9.8. Only the amide I band, between 1600 cm⁻¹ and 1700 cm⁻¹ of each spectrum was considered and analyzed on the Fityk software. A baseline was applied to each spectrum using a built-in convex hull algorithm. The baselined amide I band was deconvoluted into 9 distinct Lorentzian peaks. Peak centers were assigned based on those used in previous studies. Peak areas were integrated and area percentages were calculated as the individual peak areas over the sum of all peak areas.

As illustrated in FIG. 2B and FIG. 2D, respectively, major amide I bands were observed in both 8×KLV-Mfp5 and 8×KLV hydrogels, indicating the self-assembly of β-sheets from the zipper-forming amyloid sequences. Based on deconvolution of the amide I bands, the percentages of β-sheet content in the hydrogel were estimated to be approximately 34.6% and 39.4% for 8×KLV and 8×KLV-Mfp5, respectively. These results suggested that the addition of the amorphous Mfp5 sequence to 8×KLV did not dramatically change the self-assembly of β-sheets from the zipper-forming repeats.

### EXAMPLE 3: Mechanical Properties of 8×KLV and 8×KLV-Mfp5 Hydrogels

To evaluate the mechanical properties of the 8×KLV and 8×KLV-Mfp5 hydrogels described in Example 2, the following experiments were conducted.

Mechanical properties were measured using an MTS Criterion Model 41 universal test frame fitted with a 25 N load cell (MTS Systems Corporation, Eden Prairie, MN). To measure hydrogel tensile strength and elasticity, a dog bone-shaped protein hydrogel sample was gripped on both ends of the wider portions. Pull tests were conducted at a crosshead speed of 16 mm/min. Ultimate tensile strength was calculated as the breaking force divided by the initial rectangular cross-sectional area of the mid-section of the dogbone.

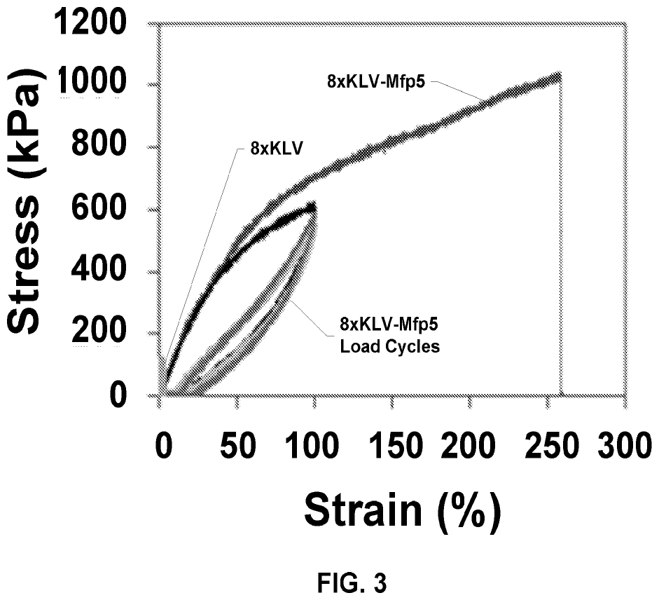

8×KLV-Mfp5 hydrogel exhibited elastic behavior, being able to stretch, on average, approximately 3 times its original gage length (strain>300. As illustrated in FIG. 3, the ultimate tensile strength of the 8×KLV-Mfp5 hydrogel was approximately 1.0±0.5 MPa, drastically higher than those of 8×KLV or other dopamine-grafted alginate-polyacrylamide hydrogels and polydopamine-polyacrylamide hydrogels (not shown). Further, the 8×KLV hydrogel was very brittle, breaking at approximately 103% strain. These results indicated that the elasticity of the 8×KLV-Mfp5 was mostly associated with the addition of the amorphous Mfp5 domain, which formed extensive and strong intermolecular interactions. Interestingly, when casting hydrogels of the Mfp5 protein in isolation under similar conditions, the Mfp5 protein aggregated without forming a hydrogel (data not shown). This observation indicated that the self-assembled β-sheets served as structural scaffolds, spatially separating Mfp5 for effective intermolecular interaction, while Mfp5 alone without the structural domain collapsed and aggregated. These results also highlighted the importance of designing semi-crystalline materials containing both structured and unstructured domains as an approach to obtain high mechanical performance.

Cyclic tensile tests were also performed to evaluate the viscoelastic properties of the 8×KLV-Mfp5 hydrogel samples described above. As illustrated in FIG. 3, a large mechanical hysteresis behavior was observed in the first pulling-relaxing cycle (black trace)) with a damping capacity of 220 KJ/m³. Without being limited to any particular theory, the large initial damping capacity appeared to arise mostly from extensive interactions between Mfp5 domains, where non-covalent bonds between neighboring Mfp5 can be sacrificed during force-loading. A high damping capacity may be desirable for materials that need to dissipate large amounts of energy created during detaching. In the second loading-unloading cycle (dark grey trace), the damping capacity decreased by 68% to 70.7 KJ/m³, suggesting that most of the sacrificed bonding from the first loading-unloading cycle was irreversible. However, the damping capacity in the third cycle (light gray trace) did not decrease, indicating that the material reached a conformational reversible equilibrium after the second loading-unloading cycle. This was likely due to the interactions between β-crystals being disrupted during the first cycle that reached an altered alignment pattern that may only be achieved with the reinforcement of the Mfp5 domains.

### EXAMPLE 4: Adhesion Properties of 8×KLV and 8×KLV-Mfp5 Hydrogels

To evaluate the bulk underwater adhesion properties of the hydrogels described in Example 2, the following experiments were conducted.

For adhesion studies, protein hydrogels were prepared as described in Example 2 above, but into square PDMS molds of dimension 10×10 mm. The hydrogels were applied on various “bottom” adherend substrates submerged in a PBS bath. While under solution, a corresponding “top” adherend substrate was applied on top of the hydrogel, such that the hydrogel was completely covered and overlapped by both adherends. Constant pressure was applied to the adherend overlap area (10 mm×10 mm) for 18 hours at 37° C. to allow the hydrogel to cure.

Adhesion properties were measured under ambient conditions using an MTS Criterion Model 41 universal test frame fitted with a 1000 N load cell (MTS Systems Corporation). Adhesion tests were conducted at a crosshead speed of 2 mm/min. The maximum force at fracture was divided by the area of the protein hydrogel (in the case of most substrates tested, the adherend overlap area) to determine the adhesion strength.

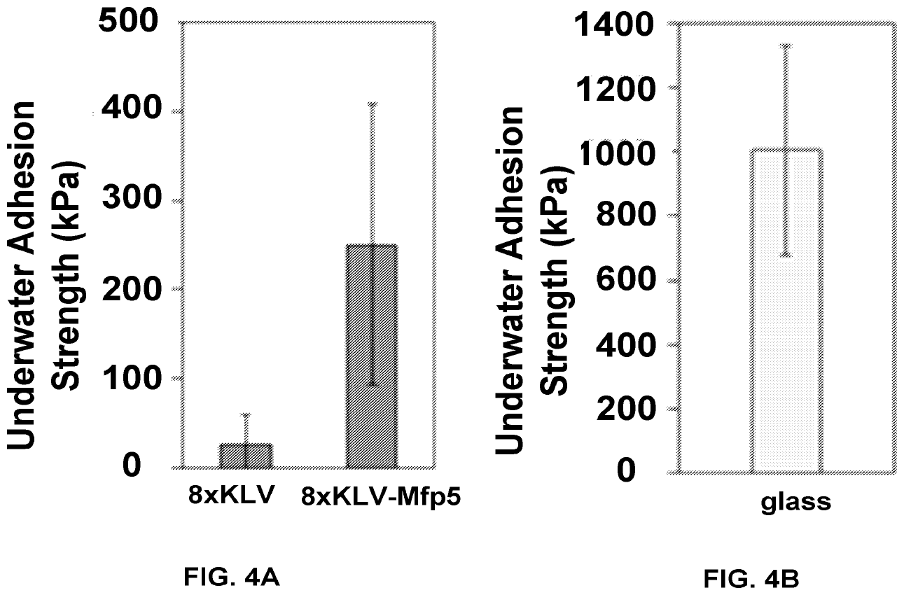

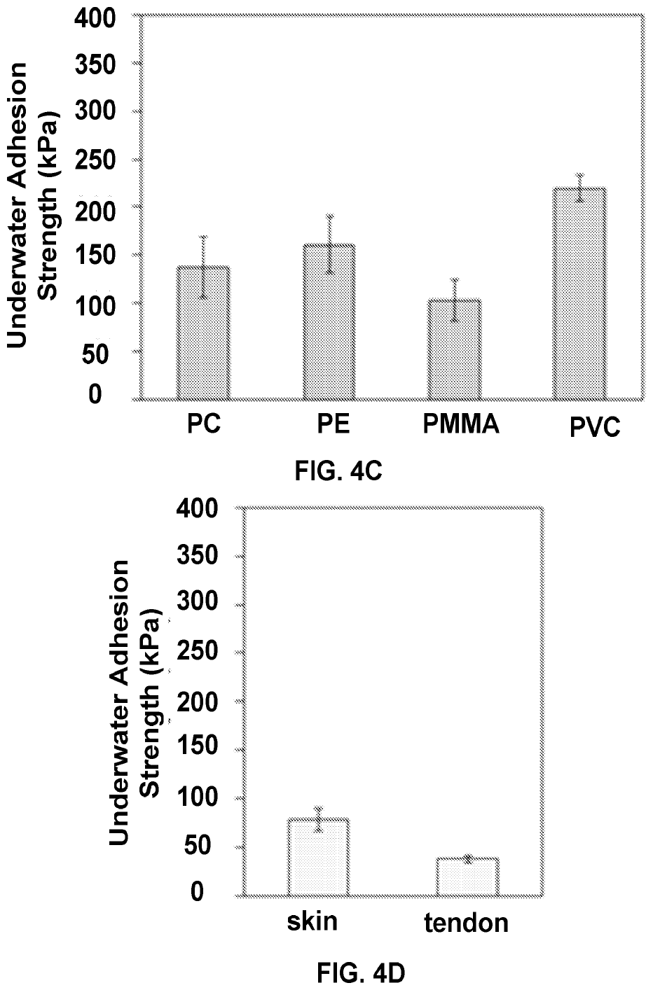

Due to its solid nature and negative buoyancy in water, the hydrogel was easily applied between adherend surfaces underwater. After curing the hydrogel underwater between two adherend bars of identical materials, the sandwiched hydrogels were subjected to single-lap shear tests in ambient conditions to evaluate adhesion strength at failure. The 8×KLV-Mfp5 protein hydrogel displayed a strong underwater adhesion on a diverse array of surfaces, including aluminum (FIG. 4A), glass (FIG. 4B), plastics (FIG. 4C), and mammalian tissues (FIG. 4D). The adhesion strength of the 8×KLV-Mfp5 protein hydrogel on aluminum (FIG. 4A) was 250±158 kPa, 9.4-fold higher than that of 8×KLV, suggesting that the surface adhesion was mostly effectuated by the Mfp5 domain of the 8×KLV-Mfp5 protein. The overall adhesion strength was also drastically higher than that of previously published values for dopamine-grafted alginate-polyacrylamide hydrogels (8 kPa) and polydopamine-polyacrylamide hydrogels (17 kPa). On glass (FIG. 4B), the 8×KLV-Mfp5 protein hydrogel displayed an average adhesion strength of approximately 1.0±0.33 MPa, which was comparable to the dry adhesion strength of some commercial adhesives and previously studied materials. On various plastic surfaces (FIG. 4C), underwater adhesion strengths between 104 to 220 kPa were obtained. Compared to glass and aluminum, the relatively lower adhesion strength on plastic surfaces was likely caused by the relatively weak hydrophobic interactions, as opposed to strong catechol-metal coordination and catechol-silica bidentate hydrogen bonding interactions. The 8×KLV-Mfp5 protein hydrogel displayed underwater adhesion strengths of 78±11 kPa and 38±3.4 kPa on porcine skin and bovine tendon (FIG. 4D), respectively, making this adhesive composition potentially useful as a surgical glue for medical applications such as tendon-bone repair.

As illustrated in FIGS. 4A, 4C, and 4D, the majority of lap shear tests exhibited adhesive failure, where the hydrogel detached completely from one surface. However, on glass surfaces (FIG. 4B), cohesive failure was observed. This observation was consistent with the higher ultimate strength of the 8×KLV-Mfp5 hydrogel (˜1 MPa), as illustrated in FIG. 3. On glass surfaces, the surface adhesion of the 8×KLV-Mfp5 hydrogel was comparable to the hydrogel cohesive strength, thus resulting in cohesive failure, in which the hydrogel was separated with pieces adhered to each surface. Interestingly, all adhesive failure was observed on the hydrogel surface that was in contact with the PDMS mold during hydrogel fabrication. Indeed, when hydrogels were removed from the PDMS mold after fabrication, the hydrogels were bound to the PDMS mold, and detaching from the PDMS mold caused partial damage to this surface.

### EXAMPLE 5: Controllable Debonding of 8×KLV-Mfp5 Hydrogels

To demonstrate controllable debonding of the hydrogels described in Example 2 from underlying adherend substrates, the following experiments were conducted.

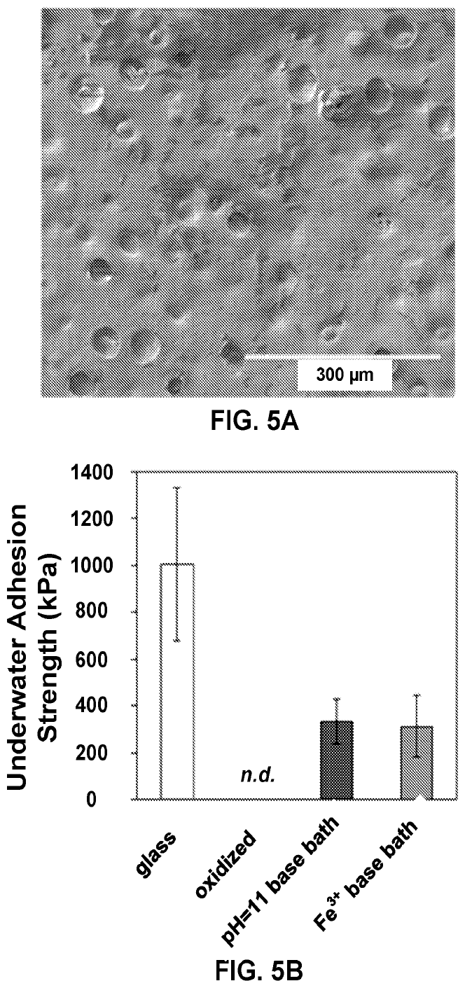

To further demonstrate the role of the catechols in the surface adhesion properties of the 8×KLV-Mfp5 hydrogels described above, bonded hydrogels were intentionally oxidized in pH 11 aqueous buffer to promote oxidation of catechol to quinone. This oxidation was associated with a yellow-to-brown color change of the hydrogel. However, as shown by SEM imaging the surface morphological features were not significantly changed before oxidation (FIG. 2C) as compared to after oxidation (FIG. 5A). When tested on glass surfaces (FIG. 5B), the oxidized hydrogel completely lost its adhesion, indicating that the bidentate hydrogen bonding between catechol and silica provided the major surface interactions.

To confirm the role of bidentate hydrogen bonding of the catechols in the 8×KLV-Mfp5 hydrogels in surface adhesion, the hydrogel-adhered glass substrates were also submerged in either a FeCl₃ solution (1 mM) or a pH 11 buffer for 8 hours. Fe³⁺ was known to form strong catechol-Fe³⁺ chelation and to debond catechols from hydrogen bonding to silica. As summarized in FIG. 5B, the adhesion strength of the 8×KLV-Mfp5 hydrogel was weakened by more than 3-fold to 312 and 334 kPa in FeCl₃ solution and pH 11 buffer, respectively. The reduced adhesion strength was likely caused by the diffusion of Fe³⁺ and base between the bonded adherend surfaces. Although oxidation was visually more apparent only at the edges of the hydrogel, the loss in adhesive interactions at these locations further compounded the stress singularities that existed at the edge of the hydrogel adhesive.

What is claimed is:

1. A hybrid protein comprising an amino acid sequence, the amino acid sequence comprising:
a. at least two zipper-forming protein sequences and at least one flexible sequence from a spider silk protein sequence, wherein each of the at least one flexible sequences is positioned between each pair of the at least two zipper-forming protein sequences to form a repeated sequence; and
b. at least one mussel foot protein sequence, each of the mussel foot proteins positioned at a C-terminus or an N-terminus of the alternating repeated sequence.

2. The protein of claim 1, wherein each of the at least two zipper-forming protein sequences is selected from a human prion sequence (SEQ ID NO:1), an αβ-crystallin sequence (SEQ ID NO:2), an islet amyloid polypeptide sequence (SEQ ID NO:3), an Aβ-amyloid sequence (SEQ ID NO:4), any fragment thereof, and any variant thereof, wherein each of the at least one flexible sequence from a spider silk protein sequence comprises a glycine-rich sequence from a Nephila clavipes MaSp1 dragline spidroin (SEQ ID NO:5), and wherein each of the at least one mussel foot protein sequences is selected from a mussel foot protein-3 Mfp3 sequence (SEQ ID NO:6), a mussel foot protein-5 Mfp5 sequence (SEQ ID NO:7), a mussel foot protein-6 Mfp-6 sequence (SEQ ID NO:6), any fragment thereof, and any variant thereof.

3.-7. (canceled)

8. The protein of claim 2, wherein the protein is an 8×KLV-Mfp5 comprising the repeated sequence and the Mfp5 sequence attached at the C-terminus of the repeated sequence, the repeated sequence comprising eight Aβ-amyloid sequences separated by the glycine-rich sequences from the Nephila clavipes MaSp1 dragline spidroin.

9. An adhesive hydrogel comprising a hybrid protein, the hybrid protein comprising an amino acid sequence, the amino acid sequence comprising:
a. at least two zipper-forming protein sequences and at least one flexible sequence from a spider silk protein sequence, wherein each of the at least one flexible sequences is positioned between each pair of the at least two zipper-forming protein sequences to form a repeated sequence, wherein each of the at least two zipper-forming protein sequences is selected from a human prion sequence (SEQ ID NO:1), an αB-crystallin sequence (SEQ ID NO:2), an islet amyloid polypeptide sequence (SEQ ID NO:3), an Aβ-amyloid sequence (SEQ ID NO:4), any fragment thereof, and any variant thereof, and wherein each of the at least one flexible sequence from a spider silk protein sequence comprises a glycine-rich sequence from a Nephila clavipes MaSp1 dragline spidroin (SEQ ID NO:5); and
b. at least one mussel foot protein sequence, each of the mussel foot proteins positioned at a C-terminus or an N-terminus of the alternating repeated sequence, wherein each of the at least one mussel foot protein sequences is selected from a mussel foot protein-3 Mfp3 sequence (SEQ ID NO:6), a mussel foot protein-5 Mfp5 sequence (SEQ ID NO:7), a mussel foot protein-6 Mfp-6 sequence (SEQ ID NO:6), any fragment thereof, and any variant thereof.

10. The adhesive hydrogel of claim 9, wherein a concentration of the hybrid protein ranges from about 25 w/v % to about 35% w/v.

11. The adhesive hydrogel of claim 9, comprising an ultimate tensile strength of at least about 1.0 MPa.

12.-13. (canceled)

14. The adhesive hydrogel of claim 10, further comprising an underwater adhesion strength of at least about 250 kPa on a metal surface, of at least about 1 MPa on a glass surface, of at least about 75 kPa on a skin surface, of at least about 35 kPa on a tendon surface, and from about 100 kPa to about 250 MPa on a plastic surface.

15.-18. (canceled)

19. The adhesive hydrogel of claim 10, further comprising an initial damping capacity of at least 220 KJ/m³, and a steady-state damping capacity of at least 70 KJ/m³.

20.-33. (canceled)

34. A method of joining at least two adherend materials, comprising positioning an adhesive hydrogel between the at least two adherend materials and maintaining the position of the adhesive hydrogel to form a plurality of bonds between the adhesive hydrogel and the at least two adherend materials, wherein the adhesive hydrogel comprises a hybrid protein and the hybrid protein comprises an amino acid sequence, the amino acid sequence comprising:
a. at least two zipper-forming protein sequences and at least one flexible sequence from a spider silk protein sequence, wherein each of the at least one flexible sequences is positioned between each pair of the at least two zipper-forming protein sequences to form a repeated sequence, wherein each of the at least two zipper-forming protein sequences is selected from a human prion sequence (SEQ ID NO:1), an αβ-crystallin sequence (SEQ ID NO:2), an islet amyloid polypeptide sequence (SEQ ID NO:3), an Aβ-amyloid sequence (SEQ ID NO:4), any fragment thereof, and any variant thereof, and wherein each of the at least one flexible sequence from a spider silk protein sequence comprises a glycine-rich sequence from a Nephila clavipes MaSp1 dragline spidroin (SEQ ID NO:5); and
b. at least one mussel foot protein sequence, each of the mussel foot proteins positioned at a C-terminus or an N-terminus of the alternating repeated sequence, wherein each of the at least one mussel foot protein sequences is selected from a mussel foot protein-3 Mfp3 sequence (SEQ ID NO:6), a mussel foot protein-5 Mfp5 sequence (SEQ ID NO:7), a mussel foot protein-6 Mfp-6 sequence (SEQ ID NO:6), any fragment thereof, and any variant thereof.

35. The joining method of claim 34, wherein the at least two adherend materials are selected independently from a metal, a glass, a plastic, and a biological tissue.

36. The joining method of claim 35, wherein the biological tissue is selected from a skin tissue and a tendon tissue.

37. The joining method of claim 34, further comprising contacting the adhesive hydrogel with an oxidizing solution to debond the adhesive hydrogel and the at least two adherend materials.

38. The joining method of claim 37, wherein the oxidizing solution is selected from a FeCl₃ solution and a pH 11 buffer.

39. The joining method of claim 38, wherein the oxidizing solution is a 1 mM FeCl₃ solution.


SEQUENCE LISTING (8 sequences)

SEQ ID NO: 1 - protein, 6 aa, Artificial Sequence
  Synthetic sequence
SNQNNF

SEQ ID NO: 2 - protein, 6 aa, Artificial Sequence
  Synthetic sequence
GDVIEV

SEQ ID NO: 3 - protein, 7 aa, Artificial Sequence
  Synthetic sequence
FGAILSS

SEQ ID NO: 4 - protein, 7 aa, Artificial Sequence
  Synthetic sequence
KLVFFAE

SEQ ID NO: 5 - protein, 28 aa, Artificial Sequence
  Synthetic sequence
GGAGQGGYGGLGSQGTSGRGGLGGQGAG

SEQ ID NO: 6 - protein, 46 aa, Artificial Sequence
  Synthetic sequence
ADYYGPKYGPPRRYGGGNYNRYGRRYGGYKGWNNGWKRGRWGRKYY

SEQ ID NO: 7 - protein, 73 aa, Artificial Sequence
  Synthetic sequence
SSEEYKGGYYPGNAYHYSGGSYHGSGYHGGYKGKYYGKAKKYYYKYKNSGKYKYLKKARKYHRKGYKYYGGSS

SEQ ID NO: 8 - protein, 99 aa, Artificial Sequence
  Synthetic sequence
GGGNYRGYCSNKGCRSGYIFYDNRGFCKYGSSSYKYDCGNYACLPRNPYGRVKYYCTKKYSCPDDFYYYNNKGYYYYNDKDYGCFNCGSYNGCCLRSGY